(19) United States
(12) Patent Application Publication
(10) Pub. No.: US 2010/0185112 A1
(43) Pub. Date: Jul. 22, 2010

(54) DEVICE FOR ANALYSING AN INFLAMMATORY STATUS OF A RESPIRATORY SYSTEM

(75) Inventors: Hans Willem Van Kesteren, Eindhoven (NL); Teunis Vink, Eindhoven (NL); Nicolaas Petrus Willard, Eindhoven (NL); Jeroen Kalkman, Utrecht (NL); Milan Saalmink, Eindhoven (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N.V., EINDHOVEN (NL)

(21) Appl. No.: 12/666,398

(22) PCT Filed: Jun. 23, 2008
(86) PCT No.: PCT/IB2008/052467

§ 371 Date: Dec. 23, 2009

(30) Foreign Application Priority Data
Jun. 27, 2007 (EP) 07111132.2

(51) Int. Cl. A61B 5/08

(52) U.S. Cl. 600/532

(57) ABSTRACT

A device (100) for measuring a concentration of NO in exhaled air is provided. The device (100) comprises a mouthpiece (11), an NO sensor (12), an airway obstruction measurement and an analysis module. The mouthpiece (11) receives the exhaled air during an exhalation. The NO sensor (12) measures the concentration of NO in the exhaled air. The airway obstruction measurement module determines an airway obstruction parameter. The analysis module (16) analyzes an inflammatory status of a respiratory system based on a combination of the measured concentration of NO and the determined airway obstruction parameter.

### TECHNICAL FIELD OF THE INVENTION

The invention relates to a device for measuring a concentration of NO in exhaled air, the device comprising a mouthpiece for receiving the exhaled air during an exhalation, an NO sensor for measuring the concentration of NO in the exhaled air and an analysis module for analyzing an inflammatory status of a respiratory system based on the measured concentration of NO.

### BACKGROUND OF THE INVENTION

Such a device is known from United States patent application US 2003/0134427. Said application describes a device for measuring NO and CO₂. The NO concentration of the exhaled air (eNO) is used as a measure for the severity of inflammation of the airways in asthma patients. The NO and CO₂ concentrations during an exhalation are measured using light absorption spectroscopy. Said device uses a single laser for scanning over a wavelength range covering a NO and CO₂ absorption. The peak value of the CO₂ concentration is known to be around 4%. The measured peak value is considered to correspond to this 4% and is used for calibrating the device. The thus obtained NO concentration is then corrected in accordance with the calibration.

The device of US 2003/0134427 uses a discard container for discarding breath provided at the beginning of the exhalation. A vacuum pump and flow controller regulate the flow rate during the measurement. Some devices are available for measuring eNO values during tidal breathing, however these devices tend to be less accurate than NO measurements under fixed flow, chiefly because of contamination by NO from the nose, the variation in flow rate and lower eNO values at higher flow rates. It is a disadvantage of the device according to US 2003/0134427 that the peak value of the CO₂ concentration is user dependent and may vary, for example, because of asthma induced airway obstruction. The uncertainty about the exact value of the peak value of the CO₂ value has a negative effect on the accuracy of the eNO measurement.

An eNO measurement is usually performed at a slight overpressure to close the soft palate and prevent contamination of the air exhaled through the mouth by NO from the nasal area. Furthermore, the exhalation flow has to be kept at a low value (typically 50 ml/s) by the person exhaling into the instrument. In this procedure the eNO plateau value during the last few seconds of the exhalation is mainly determined by the NO from the lower airway epithelium. To determine e.g. the NO from the alveoli the measurement has to be repeated at different flows.

Keeping the flow accurately at a low and fixed value is difficult for some adult patients with obstructive airway problems and especially for young children. A breathing procedure at a higher flow and preferably a larger allowed flow range or even tidal breathing is therefore attractive. At these higher exhalation flow rates, it becomes more difficult to discriminate between NO from different areas of the lower airways. Furthermore, the time-resolved eNO profile will be influenced by the gas exchange processes in the lungs which will vary according to the severity and localization of the airway obstruction.

### SUMMARY OF THE INVENTION

It is an object of the current invention to provide a device for determining an inflammatory status of a respiratory system without the disadvantages of the prior art.

According to a first aspect of the invention, this object is achieved by providing a device according to the opening paragraph, further comprising an airway obstruction measurement module for determining an airway obstruction parameter, and wherein the analysis module is arranged for analyzing the inflammatory status of the respiratory system based on a combination of the measured concentration of NO and the determined airway obstruction parameter.

The eNO profile during an exhalation consists of contributions from different areas. The airway obstruction is an important factor determining the gas exchange behavior in the airways. The inventors have seen that a simultaneous determination of the NO concentration and the gas exchange behavior of the airways leads to an improved analysis of the time course of the eNO profile and enables the determination of the inflammatory status of specific lung areas. A simultaneous determination of the eNO profile and one or more parameters derived from an obstruction measurement enable to obtain, e.g., the NO generated in the bronchi with sufficient accuracy. Because the data concerning the airway obstruction provides information about the gas exchange in the lower airways, this information facilitates obtaining accurate analyses of exhaled NO.

Depending on the exhalation condition and airway obstruction, a situation may occur wherein NO generated in the bronchi dominates or a situation wherein NO generated in the bronchi and alveoli have comparable magnitudes during part of the exhalation. When an eNO measurement is performed at a fixed exhalation flow of 50 ml/s the plateau level at the end of the exhalation is dominated by the NO generated in the bronchi. For some adult people but especially for children it is difficult to exhale at such a fixed low flow rate. An exhalation under less strict conditions makes that NO generated in other airway areas will become more relevant in the measured the eNO profile. With the device according to the invention, knowledge about the inflammatory status of different areas of the respiratory system makes a more accurate eNO analysis possible.

It is an advantage of the device according to the invention that in addition to the measure of the airway inflammation, also a measure for the airway obstruction is determined based on an easy to perform measurement. As airway obstruction is relieved by different medication than inflammation, knowing the severity of airway obstruction is advantageous for dosing medication.

In a preferred embodiment, the airway obstruction measurement module comprises a CO₂ sensor for measuring a time course of a concentration of CO₂ in the exhaled air. Such a measurement of the CO₂ concentration during an exhalation is called a capnogram.

The air that is inhaled by the user comprises 21% O₂ and close to 0% CO₂. In the lungs, part of the O₂ is transferred to the user's blood and CO₂ from the user's blood is transferred to the air in the lungs. The percentage of CO₂ in exhaled air increases during an exhalation. At the end of an exhalation, the air comprises approximately 4.5% CO₂. The shape of the capnogram is deformed when the airways are obstructed. The severity of the airway obstruction may be derived from the angles of the rising slopes of the capnogram. Furthermore, the capnogram shows the periods during which dead-space air, mixed-air and air from the alveoli is exhaled. On basis of this information part of the eNO profile as a function of time can be discarded because of contamination with NO from the nasal cavities that reached the lower airways during inhalation. This nasal NO is primarily present in the dead-space volume because, it is taken up by the tissue in the lower airways.

Alternatively, the airway obstruction measurement module comprises an O₂ sensor for measuring a time course of a concentration of O₂ in the exhaled air. As the CO₂ concentration rises, the O₂ concentration falls. During exhalation the O₂ concentration drops from 21% to 16.5%. The shape of the O₂ concentration curve is similar to the shape of the CO₂ capnogram mirrored about the X-axis and thus provides similar information on the gas exchange.

Additionally, the device may further comprise a flow or pressure sensor, which enables a variable exhalation flow measurement which is easy to perform because a wider range of flow rates can be allowed. During analysis of the metabolic gas exchange and eNO profile the flow profile is taken into account.

Preferably, the device also comprises an NO scrubber for enabling a user of the device to inhale NO-free air and/or a pressure regulator to generate overpressure during exhalation to close the soft palate.

These and other aspects of the invention are apparent from and will be elucidated with reference to the embodiments described hereinafter.

### BRIEF DESCRIPTION OF THE DRAWINGS

In the drawings:

FIG. 1 shows a device according to the invention,

FIGS. 2 and 3 show other devices according to the invention,

FIG. 4 shows a flow/pressure sensor,

FIG. 5 shows a combined gas sensing unit

FIG. 6 shows a capnogram of a healthy subject,

FIG. 7 shows a capnogram of an asthmatic subject,

FIG. 8 shows an O₂ concentration curve,

FIG. 9 shows time-resolved CO₂ and eNO curves, and

FIG. 10 shows time-resolved CO₂, eNO breath and flow curves.

### DETAILED DESCRIPTION OF THE INVENTION

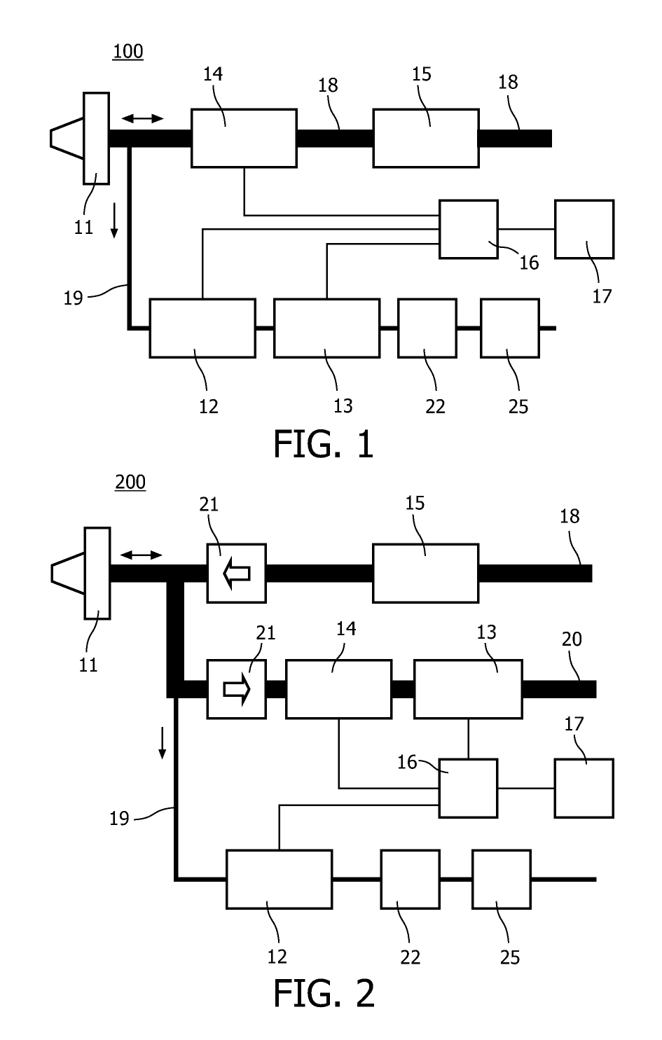

FIG. 1 schematically shows a device 100 according to the invention. The device 100 comprises an inlet or mouthpiece 11 allowing a user to exhale air through the device 100. In this embodiment, the device 100 also allows the user to inhale and exhale through the same breathing channel 18. The breathing channel 18 may comprise an NO scrubber 15 to assure that the air inhaled by the user does not comprise any NO and that all detected NO is produced in the airways of the user. The breathing channel 18 may also comprise a flow sensor 24. The function of the flow sensor 14 will be elucidated later. When exhaling into the mouthpiece 11, part of the exhaled air is directed to an analysis channel 19 using a flow restrictor 22 and a pump 25. In the analysis channel 19, a side stream NO sensor 12 and a side stream CO₂ sensor 13 are used for analyzing the exhaled breath. Optionally, both sensors 12, 13 are integrated in one combined sensor. The sensors 12, 13 may, for example, use a photo acoustic detector or optical absorption spectroscopy.

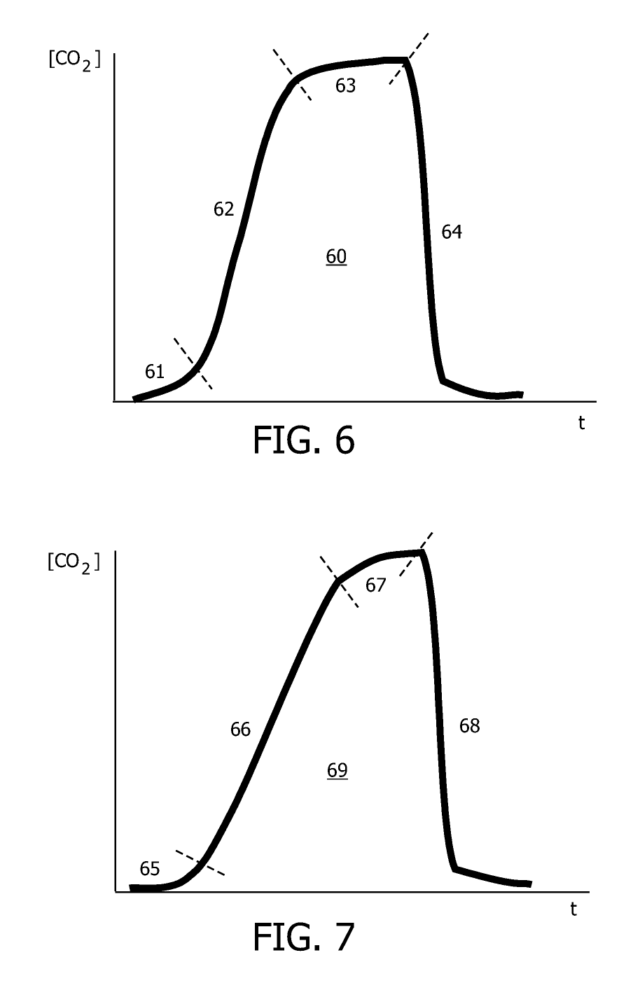

NO concentrations in exhaled air are an indication of the severity of airway inflammation. Airway obstruction may be determined using the measured CO₂ concentrations, as will be elucidated below with reference to FIGS. 6 and 7. Alternatively, the airway obstruction may be determined using a peak flow meter, a microphone or an exhalation breath temperature and/or humidity measurement module. The eNO measurement, airway obstruction information and flow data are sent to an analysis module 16. The analysis module 16 determines one or more gas exchange parameters from the measured obstruction data and uses the gas exchange parameter(s) and flow data to analyze the eNO profile. The inflammatory status determined form the eNO profile and the information about the airway obstruction is sent to user interface module 17. The obtained data may be used for advising types and dosages of medication to be used. Preferably, the analysis module 16 takes into account personal information about, e.g., sex, age, weight, normal end-tidal NO levels and normal breathing patterns.

FIG. 2 schematically shows another device 200 according to the invention. The device 200 comprises an inlet or mouthpiece 11 allowing a user to inhale and exhale air through the device 200. The device 200 allows the user to inhale through breathing channel 18. The breathing channel 18 may comprise an NO scrubber 15 to assure that the air inhaled by the user does not comprise any NO and that all detected NO is produced in the airways of the user. The breathing channel 18 also comprises a one-way valve 21. One-way valves 21 in the device take care that the inhaled and exhaled air pass though different channels of the device. The main stream exhalation channel 20 comprises a flow or pressure sensor 14 incorporating a regulating unit. The regulating unit reduces the flow in such a way that the pressure during exhalation is increased and the soft palate stays closed. In the main stream channel 20 a CO₂ sensor 13 is incorporated. Instead of a CO₂ sensor an O₂ sensor can be used or even a combination of a CO₂ and an O₂ sensor. When exhaling into the mouthpiece 11, part of the exhaled air is directed to the side stream channel 19. The side stream channel 19 comprises a flow restrictor 22 and a pump 25 which sucks a small part of the exhaled breath through this channel. In the side stream channel 19, an NO sensor 12 is used for analyzing the exhaled breath (eNO).

The time-resolved eNO, CO₂ (or O₂) and pressure/flow data are sent to analysis module 16. During an exhalation the flow/pressure data may be used to give the user feedback on required levels of exhalation force via the user interface module 17. The analysis module 16 analyses the measured eNO on basis of the flow rate and metabolic gas exchange derived from the CO₂/O₂ curves. The obtained data may be used for reporting on the inflammatory and airway obstruction status of the lower airways.

For application as a diagnostic or research device the measurement can be performed at different flow/pressure settings of the unit 14 to derive more detailed information on the inflammatory status of the lower airways.

For a personal monitoring device, the analysis module 16 may take into account personal information about, e.g., sex, age, weight, and personal reference levels for inflammation and obstruction. Advice may be provided concerning dosages of medication to be used.

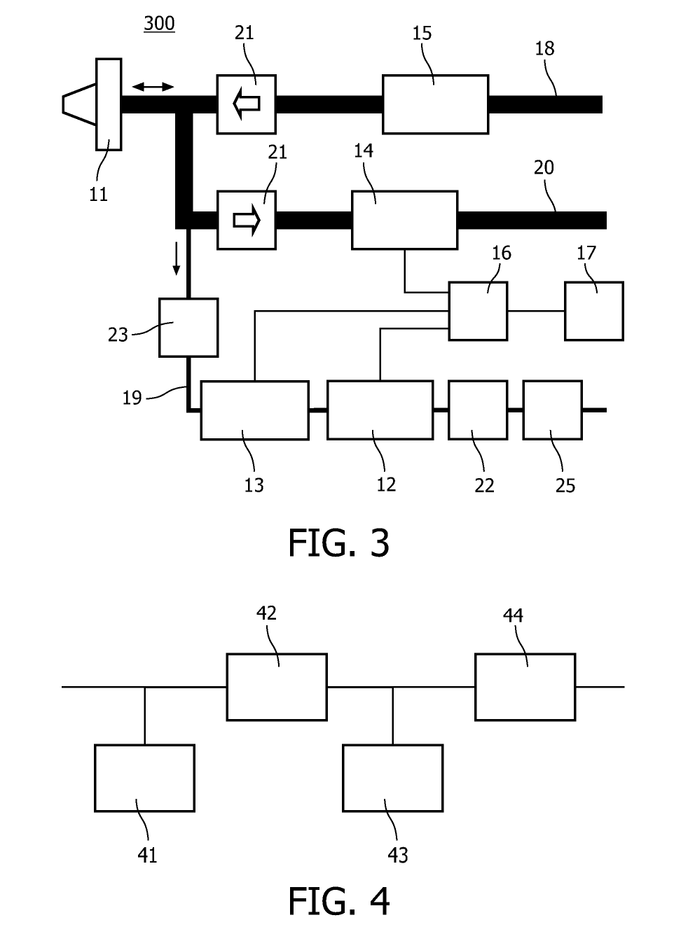

FIG. 3 schematically shows a device 300 according to the invention. In the side-stream a converter unit 23 is incorporated which converts NO in the exhaled breath into NO₂. The converter has a low volume and fast conversion rate so the time-resolved NO₂ profile at the outlet follows closely the time-resolved eNO profile. The side stream channel 19 comprises a NO₂ detection module 12, a CO₂ or O₂ detection module, 13, a flow restrictor 22 and a pump 25. In the event that the CO₂/O₂ sensor is situated behind the converter unit 23 it is essential that the converter 23 does not influence the CO₂/O₂ profile and concentration. Alternatively the CO₂/O₂ sensor may be placed in front of the converter 23 in the side stream 19. However, placement behind the converter allows for integrating of the gas sensing modules 12 and 13. Optical absorption spectroscopy allows time-resolved detection of O₂, CO₂, NO and NO₂ with accuracies as required for the breath analysis application. The device 300 may be based on a combined gas sensing unit for NO₂ and O₂ which both show absorptions in the visible wavelength range. Alternatively the NO₂ sensing is carried out in the visible wavelength range and CO₂ sensing in the near-infrared.

FIG. 4 shows an example of an implementation of the flow or pressure sensor 14. The flow or pressure sensor 14 incorporates a fixed restriction 42 with small flow impedance. This fixed restriction 42 generates a pressure drop over the fixed restriction 42. Using pressure sensors 41, 43 on both sides the pressure drop is measured and the gas flow passing this restrictor is determined. After the small flow-impedance flow-pressure sensor a higher flow-impedance flow-pressure regulator 44 is placed. This flow-pressure regulator 44 incorporates for instance a pressure sensitive spring construction and variable throughput hole. This flow-pressure regulator 44 takes care that the overpressure during exhalation is sufficient to keep the velum closed in the flow-range of the measurement.

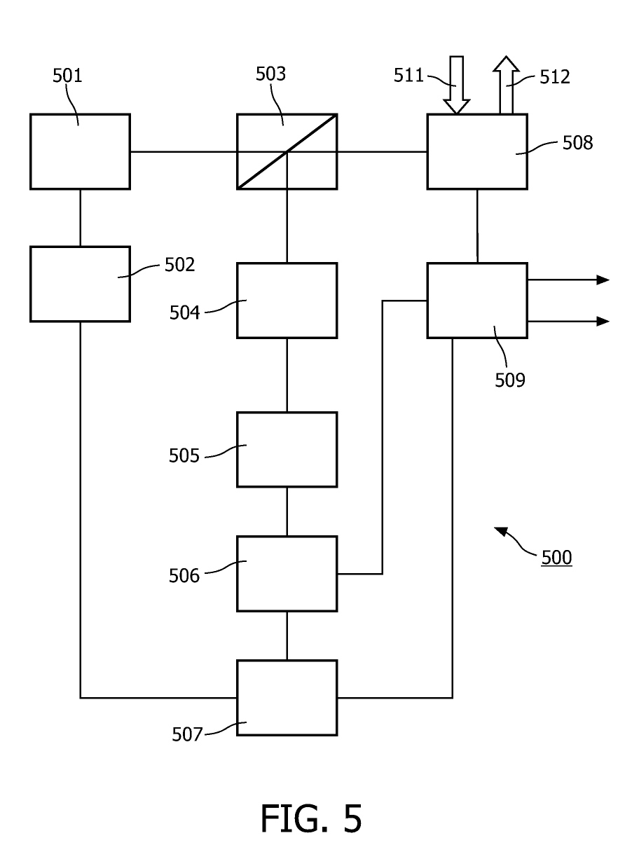

FIG. 5 shows a gas sensor 500 for simultaneous detection of two gases. A first light source 501 generates light with a wavelength that corresponds to the absorption of a first gas, e.g. NO or NO₂. A second light source 504 generates light with a wavelength that corresponds to the absorption of a second gas, e.g. CO₂ or O₂. The light sources are driven by driver units 502 and 505. The light beams are combined using, e.g., a semi-permeable mirror 503 and enter a photoacoustic gas detection unit 508 with a small sensing volume. Photo-acoustic detection offers a real time response to changes in the gas concentration. One driver unit 505 is controlled by a frequency generator 506. The other driver unit is modulated at the same frequency but with a 90 degree phase shift 507. The light is amplitude modulated at a frequency corresponding to an acoustic resonance of the detection unit 508 to improve sensitivity. This enables simultaneous detection of photoacoustic signals corresponding to both gases. The acoustic signal from the gas detection unit 508 is led to a lock-in amplifier 509 where the signal is demodulated at the reference frequency with a phase of 0° respectively 90° to obtain the concentrations of both gases.

Airway obstruction in asthma is reversible and a result of the inflammation of the lower airways. An increase in the severity of inflammation due to exposure to allergens will generally result in an increased airway obstruction. It typically takes a number of days before the severity of the obstruction increases. In COPD the obstruction is less variable but inflammation can still vary over time. Steroids, also called corticosteroids are an important type of anti-inflammatory medication. They make the airways less sensitive and less likely to react to triggers. Bronchodilators relieve the obstruction by relaxing the muscle bands that tighten around the bronchi.

NO is generated at increased concentrations in inflamed areas. Potential sources of exhaled NO are the lower airway epithelium, the upper airway (nasal) epithelium, the alveolar epithelium and the vascular endothelium. The gas exchange mechanisms for these different sources vary. The gas-phase NO concentration from the lower airway epithelium is flow-dependent while the NO coming from the alveoli is flow-independent and resembles in that respect the CO₂ gas exchange mechanism.

FIG. 6 shows a capnogram 60 of a healthy subject. The capnogram 60 comprises an exhalation phase 61, 62, 63 and an inhalation phase 64. During the exhalation phase, the CO₂ concentration detected by the CO₂ sensor 13 rises. During the inhalation phase 64, the CO₂ concentration rapidly falls to zero. The exhalation comprises three different phases 61, 62, 63. In a first phase 61, the user mainly exhales air from the mouth, which air has not been in the lungs and therefore comprises very little CO₂. The air exhaled in this first phase 61 is called dead-air. In the following second phase 62, dead air is mixed with air that has been in the alveoli (sites of gas exchange between lungs and blood) has picked up some CO₂ from the blood. The CO₂ concentration in the mixed air increases during the second phase 62, until it comes close to the end-tidal value of 4.5%. During the end-tidal phase 63, nearly all air comes from the alveoli.

FIG. 7 shows a capnogram 69 of an asthmatic subject. For asthmatic patients, the shape of the capnogram 69 is influenced by airway obstruction and unequal emptying of the alveoli. The angles of the rising slopes 66, 67 of the capnogram 69 form a measure of the severity of the airway obstruction. These slopes are related to the spread in gas exchange rates of the alveoli. When the airways are obstructed, CO₂ levels in the exhaled air rise slower than when the airways are not obstructed and the plateau region 67 is shorter. Additionally, the end-tidal CO₂ concentration may be lower than in healthy patients. According to the invention, information from the capnogram 69 is used to obtain more relevant information from the time-resolved eNO profile during an exhalation. In principle a peak-flow measurement can also be applied to determine the airway obstruction. Because the variations in peak-flow values correlate reasonably well with changes in the slopes of the capnogram, an estimate of the spread in alveolar gas exchange rates can be obtained on basis from a peak-flow measurement.

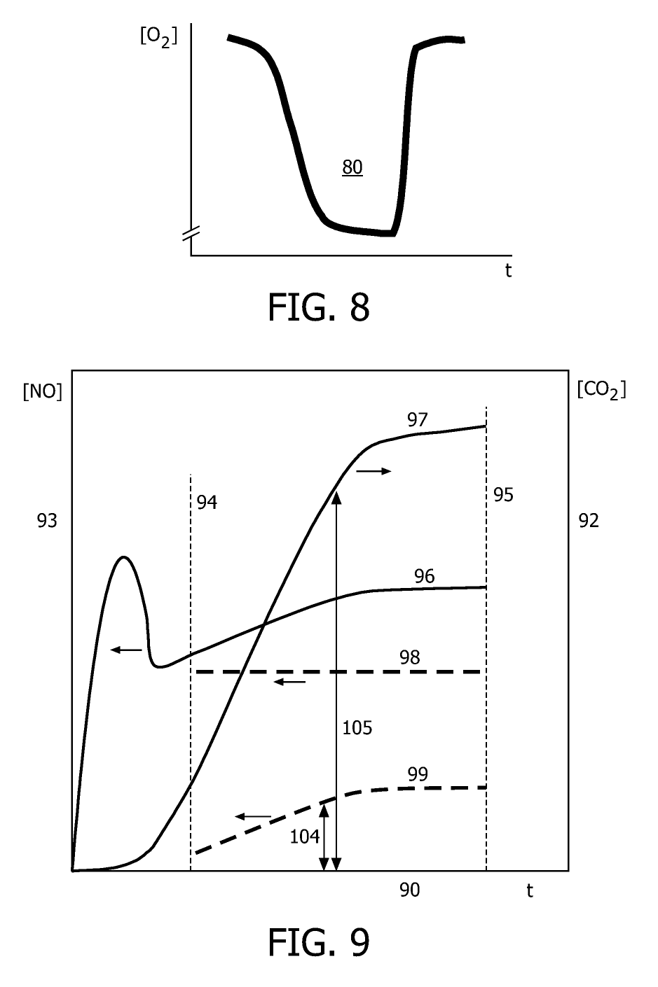

FIG. 8 shows an O₂ concentration curve 80. In the device 10 of FIG. 1, the CO₂ sensor 13 may be replaced or accompanied by an O₂ sensor. During exhalation, the O₂ concentration drops from 21% to approximately 16.5%. The O₂ concentration drops because O₂ is transferred from the air in the lungs to the blood and CO₂ is transferred from the blood to the air in the lungs. The shape of the O₂ concentration curve is similar to the shape of the CO₂ capnogram mirrored about the X-axis and thus provides similar information about (partial) obstruction of the alveoli.

FIG. 9 shows an exemplary measurement of the NO concentration 93 and CO₂ concentration 92 as a function of time 90. The measurement can be carried out with a device 200 as described before. For the measurement described here, the flow is kept at a constant value. The primary data consist of the time-resolved NO concentration 96 and time-resolved CO₂ concentration 97. For the analysis only the time span in-between the lines 94 are 95 is considered. The initial part of the exhalation until the time point corresponding to line 94 is discarded because the dead-air space can be contaminated during inhalation with some air containing NO from the nasal cavities. In the lower parts of the airways this nasal NO contamination is taken-up by the airways. When no scrubber is used during inhalation this peak can further increase. Preferably the capnogram 97 is used to determine the appropriate positioning of line 94, for instance the first bent in the capnogram or the point where the CO₂ concentration passes a certain value. Line 95 corresponds to the end of the exhalation. In-between the lines 94 and 95 the NO concentration is considered to be build up of a constant contribution from the bronchi 98 because the flow is fixed and a varying contribution 99 from the alveoli. In a simple model the latter is a constant fraction 104 of the CO₂ concentration 105. A data fitting procedure will then yield the alveolar and bronchial contribution to the exhaled NO. An advantage of the above described procedure is that the alveolar and bronchial contribution can be determined in a single experiment when the flow condition is chosen appropriately.

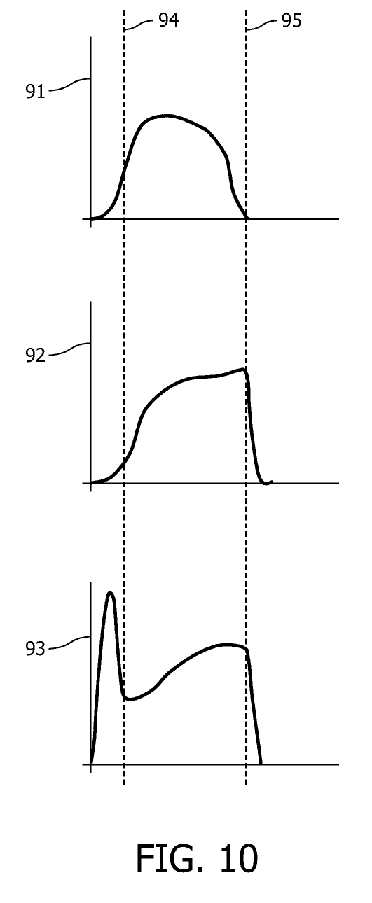

FIG. 10 shows an exemplary tidal-breathing measurement with a device 100 where the flow 91, CO₂ concentration 92 and NO concentration 93 are monitored. After discarding the “contaminated” part of the eNO profile, the remaining profile in between lines 94 and 95 is analyzed in terms of a flow dependent NO part and a NO part that follows the behavior of the metabolic CO₂-gas exchange. In its simplest form the NO generated in the bronchi follows an inverse flow dependence, while the NO from the alveoli is considered to be flow-independent and being proportional to the CO₂ concentration. On basis of this model the eNO profile in-between line 94 and 95 is fitted and two parameters obtained describing the inflammatory status of the alveoli and bronchi. More complicated dependences than described above can of course be implemented. To increase the accuracy of the parameter fitting it is possible to perform eNO measurements during a number of subsequent tidal breathings. In a personal monitoring system a number of the parameters can be set to individual values. For asthmatic persons, the bronchial NO is expected to vary according to the severity of environmental inflammatory triggers while the alveolar contribution will show less variation. In that case an accurate value for the alveolar contribution can be determined once using the measurement procedure as described for FIG. 9. If necessary a range of exhalation flow rates can be used to further improve accuracy. For subsequent monitoring of the inflammatory status of the bronchi on a regular basis a tidal-breathing apparatus is used where the alveolar contribution is set as a fixed parameter. The advantage over current measurement procedures is that the bronchial NO can be determined under e.g. tidal breathing conditions, which is easier for the customer.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer. In the claims enumerating several means, several of these means may be embodied by one and the same item of hardware. The mere fact that certain measures are recited in mutually different dependent claims does not indicate that a combination of these measures cannot be used to advantage.

1. A device (100) for measuring a concentration of NO in exhaled air, the device (100) comprising:
a mouthpiece (11) for receiving the exhaled air during an exhalation,
an NO sensor (12) for measuring the concentration of NO in the exhaled air,
an airway obstruction measurement module for determining an airway obstruction parameter, and
an analysis module (16) for analyzing an inflammatory status of a respiratory system based on a combination of the measured concentration of NO and the determined airway obstruction parameter.

2. A device (100) as claimed in claim 1, wherein the airway obstruction measurement module comprises a CO₂ sensor (13) for measuring a time course of a concentration of CO₂ in the exhaled air.

3. A device (100) as claimed in claim 1, wherein the airway obstruction measurement module comprises an O₂ sensor for measuring a time course of a concentration of O₂ in the exhaled air.

4. A device (100) as claimed in claim 1, further comprising a flow or pressure sensor (14) for measuring an exhalation flow.

5. A device (100) as claimed in claim 1, further comprising an NO scrubber (15) for enabling a user of the device (100) to inhale NO-free air.

6. A device (100) as claimed in claim 1, further comprising a pressure regulator (44) to generate overpressure during exhalation to close the soft palate.
